(19) United States
(12) Patent Application Publication
(10) Pub. No.: US 2015/0079597 A1
(43) Pub. Date: Mar. 19, 2015

(54) METHOD FOR CAPTURING AND CONCENTRATING A MICROORGANISM IN A BIOLOGICAL SAMPLE

(71) Applicant: BIOMERIEUX, Marcy I'Etoile (FR)

(72) Inventors: Jean-Pierre Flandrois, Lyon (FR); Jean-Claude Raymond, Bessenay (FR); David Mosticone, Saint Consorce (FR); Pradip Patel, Grezieu la Varenne (FR); Thierry Sofia, Marcy (FR)

(21) Appl. No.: 14/366,082

(22) PCT Filed: Jan. 8, 2013
(86) PCT No.: PCT/FR2013/050035

§ 371 Date: Sep. 22, 2014

(30) Foreign Application Priority Data
Jan. 10, 2012 (FR) 1250260

(51) Int. Cl. G01N 33/569

(52) U.S. Cl. CPC G01N 33/56916; G01N 2333/245
USPC 435/6.12; 435/7.1; 435/7.37

(57) ABSTRACT

The present invention relates generally to the field of analysis, for example biological analysis. More specifically, the present invention relates to a method for capturing and concentrating at least one microorganism or at least one protein secreted by a microorganism that may be present in the sample placed in a container, the method including the following steps: a) in the container, bringing the sample into contact with a culture medium and a sponge capable of capturing the microorganism(s) or the protein(s) secreted by at least one microorganism to be detected, functionalized with a binding partner of at least one microorganism or of at least one secreted protein; b) placing the container in suitable conditions allowing growth of the microorganism or microorganisms; and c) repeatedly compressing and decompressing the sponge while it remains in contact with the medium.

The present invention relates generally to the field of analysis, for example biological analysis. More specifically, the present invention relates to a method for capturing and concentrating at least one microorganism or at least one protein excreted by a microorganism that may be present in a sample.

This method is particularly applicable to pathogenic microorganisms contained in complex media, in low concentration.

Microbiological analysis requires accurate methods, in which the time to obtain the result must be as short as possible.

In the medical field, it is necessary to predict and diagnose the risk of infection: the quicker and more accurate the diagnosis, the more effective is patient management, and the risk of transmission is minimized. The approach is similar for animal health.

There is an identical range of problems in the food industry. However, a distinction is made between:
pathogenic microorganisms and their toxins, investigation for which applies to raw materials, intermediates, marketed finished products,
non-pathogenic microorganisms, used as indicators of quality of the production process, from the raw materials to the finished products, throughout the production chain, and
bacteria of technological interest, such as ferments.

In the context of the presence of pathogenic microorganisms, rapid and accurate detection of presumed contaminants makes it possible to control them and thus apply corrective actions.

So as to be able to detect the presence of these microorganisms, it is necessary to take large enough samples in order to be certain of recovering a minimum quantity of microorganisms. It is then necessary to increase their concentration, isolate them and identify them.

Microbiological analysis therefore requires one or more steps of pre-enrichment and/or enrichment, one or more steps of detection, and one or more steps of counting the microorganisms. For particular applications such as microbiological control in the food industry, a confirmation step may also be required, in order to comply with the current standards in this field.

At present, there is no method available for directly detecting a target microorganism in a large initial amount of sample, without employing an enrichment step.

The step of pre-enrichment and/or enrichment requires selective or non-selective culture media, the aim of which is to promote growth of the target microorganisms in the biological or environmental samples, while limiting growth of the non-target flora. In this way, the target population, which is often present at low levels relative to the subsidiary flora present in foodstuffs, is amplified. Enrichment therefore makes it possible to obtain a concentration of the microorganisms of 10⁴ to 10⁶ cells/millilitre, allowing them to be detected. Culture may also revitalize the microbial cells that may have been stressed during industrial processes.

The media are often used in containers of the sterile plastic bag type, in which they are brought into contact with the environmental or food samples, allowing resuspension and enrichment of the microorganisms being sought. This step is necessary in order to meet the requirement of revealing the potential initial presence of at least one target microorganism in a very variable amount of sample, optionally very large, e.g. 25 grams (g) to 375 g diluted in 225 to 3375 millilitres (mL) in the culture medium. At the end of this enrichment step, an aliquot (from 5 microlitres (μL) to 5 mL) is taken for carrying out the step of detecting the target microorganisms. Now, in this aliquot, it is necessary to have a sufficient quantity of target microorganisms to ensure systematic detection thereof.

The detection step is based historically on culture of the microorganisms on agar media, for detecting the metabolic characters of the microorganisms being sought. Specific enzyme substrates may be used. These enzyme substrates are generally made up of two parts, a first part specific to the enzyme activity to be revealed, also called the target part, and a second part serving as marker, called the marker part, generally consisting of a chromophore or a fluorophore. By selecting these substrates, depending on whether there is reaction or not, it is possible to characterize the nature of a microorganism or differentiate different groups of microorganisms. Thus, the appearance or disappearance of a coloration or of a fluorescence will be the signature of a genus or of a type of microorganism. In this respect, the use of chromogenic media allows simultaneous detection and identification of the microbes being sought. It simplifies the process and greatly reduces the delay in obtaining the result. We may mention, as a concrete example, the applicant's ChromID® media. These chromogenic media are based on the detection of metabolic characters specific to the microbes being sought, for example beta-glucuronidase enzyme activity for Escherichia coli.

Immuno-assays constitute another of the technologies used for the detection test. They make use of the immunogenic characteristics of the microorganisms being sought. Non-exhaustively, we may mention the ELISA techniques (Enzyme-Linked ImmunoSorbent Assay), competitive or of the sandwich type.

Finally, the techniques of molecular biology, based on the genomic characters of the microorganisms being sought, are also employed for detecting and identifying the target microorganisms. We may mention, as an example, the classical amplification techniques such as PCR (Polymerase Chain Reaction) and NASBA (Nucleic Acid Sequence Based Amplification), which may be coupled to real-time detection techniques known by a person skilled in the art.

As for the confirmation step, it is more particularly associated with microbiological analysis in the food industry. In fact, when the result of the methods developed previously is positive, it is necessary to confirm the presence of the pathogen being sought. This requires a complementary test and the use of a detection principle different from that used in the first analysis. The techniques described above may be used again for confirmation.

The complete and accurate identification of a microorganism in a sample therefore requires a succession of several steps: enrichment, detection and confirmation. Standardization of the tests used routinely has allowed the methods of detection to be automated. The steps prior to detection are the most time-consuming, conventionally taking 6 to 24 hours. These steps therefore require the elaboration of novel technical solutions for obtaining the results rapidly, starting from the initial sample.

Methods of capture of microorganisms carried out after the enrichment step have already been described. Thus, filters are widely used. However, they are not very suitable for treating large-volume samples. They are more used for samples containing little food matter, such as water or beverages. In fact their efficacy is greatly reduced once the sample is complex, leading to obstruction of the pores of the filters. More sophisticated filters have been developed. Thus, document WO 2009/018544 describes a multilayer filter notably comprising a layer of porous microbeads.

Centrifugation and flocculation are also used. These methods, although listed, have several major drawbacks. They are traumatizing for the microorganisms that are to be concentrated. Thus, at the end of these treatments, more than 50% of the microorganisms disappear or have been destroyed.

They also have low selectivity: the centrifuged or flocculated microorganisms are accompanied by contaminants that will inhibit the subsequent methods of analyses such as the polymerase chain reaction. These contaminants may also distort the analyses.

Document WO 2005/069005 describes a method of extracting the microorganisms using a sponge, comprising steps of compression/decompression of the sponge in a washing buffer to facilitate detachment of the substances not required and finally a step of elution in a new container in order to recover the targeted analyte. This method does not envisage an enrichment step simultaneous with capture of the target microorganisms.

More complex systems have also been described. This applies for example to the Pathatrix system developed by the company Matrix MicroScience. This system consists of a capture step, comprising antibodies immobilized on magnetic beads, on which the sample is circulated. This method also does not envisage an enrichment step simultaneous with capture of the target microorganisms. Another drawback of this system is that the capture surface of the magnetic beads is still limited.

There is therefore a real need for a new method of preparing the sample that allows the time taken for analysis of the sample to be reduced while allowing rapid and effective detection of the target microorganisms.

One aim of the present invention is therefore to provide a method that improves the capture and concentration of the microorganisms or of the proteins excreted.

Another aim of the present invention is to provide a method thus permitting better detection of the target microorganisms by revitalization and efficient growth of these microorganisms.

Another aim is to propose a method for capturing and concentrating microorganisms that is suitable for the analysis of large-volume samples, obtained from complex media. “Complex media” means media containing biological, organic and mineral materials in suspension, and notably the target microorganisms.

Another aim of the present invention is to propose a method for capturing and concentrating microorganisms which may easily be coupled to the various existing techniques for detection and identification.

In this connection, the invention relates to a method for capturing and concentrating at least one microorganism or at least one protein secreted by at least one microorganism that may be present in a sample placed in a container, said method comprising the following steps:
a) in the container, bringing said sample into contact with a culture medium and a sponge capable of capturing the microorganism(s) or the protein(s) secreted by a microorganism to be detected, functionalized with a binding partner of at least one microorganism or of at least one excreted protein,
b) placing the container in suitable conditions allowing growth of the microorganism or microorganisms,
c) repeatedly compressing and decompressing the sponge while it remains in contact with said medium.

In the sense of the present invention, the term “microorganism” covers Gram-positive or Gram-negative bacteria, yeasts, moulds and, more generally, unicellular organisms, invisible to the naked eye, which can be manipulated and multiplied in the laboratory.

The present invention also makes it possible to detect the proteins secreted by microorganisms. We may mention for example the detection of toxins secreted by Staphylococcus aureus.

“Sample” means a small portion or small amount isolated from one or more entities for analysis. It may have undergone a previous treatment, such as mixing, dilution in a liquid medium or else grinding notably if the entity is solid.

The samples may be of food-industry, environmental or clinical origin.

Among the samples of food-industry origin, we may mention non-exhaustively a sample from milk products (yoghurts, cheeses, etc.), meat, fish, eggs, fruit, vegetables, water, drinks (milk, fruit juices, soda, etc.). These samples of food-industry origin may also originate from sauces or from ready meals. A food sample may finally be obtained from feed intended for animals, such as notably from animal meal.

We may also mention samples connected with the environment, such as samples collected from the surface, water, or air.

Samples of clinical origin may correspond to samples of biological fluids (whole blood, serum, plasma, urine, cerebrospinal fluid), from faeces, samples collected from the nose, throat, skin, wounds, organs, tissues or isolated cells etc.

The sample is brought into contact with a culture medium allowing growth of the microorganisms. “Culture medium” means a medium comprising all the elements necessary for the survival and/or growth of the microorganisms. The culture medium may contain optional additives, for example: peptones, one or more growth factors, carbohydrates, one or more selective agents, buffers, one or more gelling agents etc. This culture medium may be in the form of liquid, or of gel ready for use, i.e. ready for seeding in a tube, flask or Petri dish. Thus, one of the advantageous aspects of the invention is growth of the microorganisms simultaneously with capture thereof The growth of the target microorganisms then takes place directly on the sponge. As the microorganism is less in contact with the subsidiary microbial flora potentially present in the mixture, the growth of the microorganism is improved and its localization is facilitated. The concentration of the microorganism or of the protein secreted is therefore improved.

“Sponge” means a compressible solid support formed from a porous material. It may be of natural origin, artificial or synthetic. The shape of the sponge and the pore size may vary depending on the desired applications.

In a particular embodiment, the functionalized sponge is immersed in the culture medium.

In another embodiment, the culture medium circulates through the sponge.

According to the method of the present invention, the sponge is compressed and decompressed repeatedly. The volume of the culture medium brought into contact with the sponge is increased by the action of compression/decompression. Thus, this step allows acceleration of capture of the analyte in a volume far greater than that of the sponge itself

The compression/decompression may be done manually or mechanically by any means known by a person skilled in the art.

The binding partner recognizing the microorganism or the protein secreted is immobilized specifically or non-specifically on the sponge. Preferably the binding partner is selected from proteins, antibodies, antigens, aptamers, phages, phage proteins, nucleic acids or carbohydrates. It may also be of polymeric nature (chitosan, poly-L-lysine, poly-aniline) so as to allow non-specific capture of the cells.

The term antigen denotes a compound capable of being recognized by an antibody whose synthesis it has induced by an immune response.

The term antibody includes the polyclonal or monoclonal antibodies, the antibodies obtained by genetic recombination and antibody fragments.

Phages, or bacteriophages, are viruses that only infect bacteria; they are also called bacterial viruses.

Fixation on the sponge may correspond to direct or indirect immobilization: direct immobilization means fixation by covalency or passive adsorption. Direct immobilization may be effected by means of a ligand fixed chemically on the sponge. Indirect immobilization means ligand/antiligand interaction between a ligand fixed on the antigen, antibody or phage (more widely, the functional compound) and the antiligand or complementary ligand fixed on the sponge.

Ligand/antiligand pairs are well known by a person skilled in the art, and we may mention for example the following pairs: biotin/streptavidin, hapten/antibody, antigen/antibody, peptide/antibody, sugar/lectin, polynucleotide/complementary polynucleotide.

A water-soluble compound derived from a homopolymer or copolymer of maleic anhydride such as those developed by the applicant in the granted patent EP 0 561 722 may also be used for immobilizing a biological molecule.

Advantageously, the binding partner is bound to the sponge via a dopamine polymer.

According to one embodiment, the method according to the present invention comprises an additional step consisting of transferring some or all of the mixture consisting of said sample, the culture medium, the sponge and optionally a developer system, from the container, then called main container, to at least one second, so-called secondary container.

It is optionally possible to carry out a secondary enrichment in the secondary container, by adding the nutrients and selective agents ad hoc to said secondary container beforehand. This secondary enrichment makes it possible to increase the population of the target microorganism(s) relative to that of the non-target microorganisms, which improves the specificity.

One or more washing steps may take place prior to the detection step and after step c).

According to one embodiment, a developer system able to permit detection is put in contact in the main container or the secondary container. “Developer system” means any molecule capable of coupling with the microorganisms, the secreted proteins or the binding partners of said microorganisms and making it possible, by their properties of transduction (notably fluorescence, coloration, radioactivity), to reveal the presence of said microorganisms.

The detection step may be carried out in real time or at the end of the growth phase of said microorganism or said microorganisms.

Thus, the method according to the present invention may comprise an additional step of detecting the microorganism or secreted protein bound to the binding partner.

According to one embodiment, the sponge is transferred to a secondary container containing a culture medium which may in addition contain a substrate allowing detection of an enzymatic or metabolic activity of the target microorganisms owing to a signal that is detectable directly or indirectly. For direct detection, this substrate may be bound to a part serving as a fluorescent or chromogenic marker. For indirect detection, the culture medium according to the invention may comprise in addition a pH indicator, sensitive to the change in pH induced by the consumption of substrate and revealing growth of the target microorganisms. Said pH indicator may be a chromophore or a fluorophore. We may mention, as examples of chromophores, neutral red, aniline blue, bromocresol blue. The fluorophores comprise for example 4-methylumbelliferone, the derivatives of aminocoumarin or the derivatives of resorufin. Another example of indirect detection may consist of the use of latex specifically sensitized with an antibody directed against the analyte being sought.

According to one embodiment, the developer system is a nonspecific substrate internalized by the microorganism or microorganisms to be detected. According to a particular example, the developer system is based on the reduction of TTC by the microorganisms. Simultaneously with growth, TTC (colourless in its unreduced form) is internalized by said microorganisms, then reduced by the latter to triphenylformazan (red) thus staining said microorganisms red and then allowing them to be revealed on the sponge. The method of direct detection in real time of microorganisms in a food sample, during the incubation period, is carried out by optical reading of the sponge, which may or may not be automated. Incubation may be carried out at temperatures between 25 and 44° C. for 6 to 48 h. Thus, once there is effective capture of a certain quantity of stained target microorganisms (in the case of a positive sample), there is a change in the optical properties of the sponge through the appearance of a red coloration on the latter (i.e. transduction of the biological signal). This coloration of the capture substrate is then detectable by eye or measurable using an automatic reading device such as a camera. To facilitate reading, it is preferable that the sponge is no longer in contact with the culture medium. For this purpose, it may be envisaged for example to tilt the homogenizing bag. As explained above, the reading may be taken at the end point, at dots or in real time.

According to another embodiment, the developer system is a cellular stain of the microorganism or microorganisms to be detected.

The detection step may be carried out using a means selected from optical detecting means, magnetic detecting means, electrochemical detecting means, electrical detecting means, acoustic detecting means, and thermal detecting means.

According to one embodiment, the detection step is carried out directly on the sponge. According to a particular embodiment, the sponge is compressed during the detection step, thus permitting amplification of the signal.

In another embodiment, the method according to the invention comprises an additional step of elution of the microorganism and/or of the secreted protein captured by the sponge. This step takes place before the detection step. This step is particularly advantageous for the methods of detection using immunochemical techniques, amplification of nucleic acids, mass spectroscopy or Raman spectroscopy.

The examples presented below have the aim of presenting various embodiments of the method according to the invention and the results obtained. They do not limit the invention in any way.

### EXAMPLE

### Example 1

### Preparation of a Capture Substrate Sensitized with at Least One Binding Partner Specific to the Target Microorganism Permitting Capture of the Target Microorganisms

A capture substrate, a sponge, consisting of a cube of polyurethane foam with an internal capacity of about 2 ml is sensitized as follows:

The cube of polyurethane foam (sponge) is “filled” by cycles of compression/decompression with a solution of 3,4-dihydoxyphenylalanine at 2 g/1 in Tris-HCl buffer pH 8.5 and then remains immersed in said solution at room temperature for 18-24 h.

The sponge is then rinsed in sterile demineralized water 3 times by compression/decompression cycles.

The sponge is then immersed for two hours at room temperature in a solution of specific binding partners (1 μg/mL to 40 μg/mL) in PBS buffer pH 7.2.

The sponge is finally passivated in a solution of BSA in Tris-Maleate buffer at pH 6.2, for 1 hour at room temperature.

The sponge is then rinsed in PBS buffer pH 7.2, 3 times by cycles of compression/decompression.

The sensitized cube of foam may be used for capturing microorganisms or stored at 2-8° C. with a view to later use.

### Example 2

### Optical Detection of the Presence of Escherichia coli O157:H7 in a Food Sample by Using a Sensitized Sponge

The aim of this experiment is to detect directly, using a sensitized substrate as described above, the presence of the target bacterium E. coli O157:H7 in a food sample during enrichment.

As detailed below, detection is carried out during the incubation period by immersing the capture substrate sensitized with an anti-E. coli O157:H7 recombinant phage protein in a homogenizing bag containing the food sample, diluted to 1/10th in the reaction mixture.

Two samples are prepared as follows:
Sample A: In a homogenizing bag, 25 g of minced steak contaminated with 5 colony-forming units (CFU) of E. coli O157:H7 are resuspended in 225 mL of BPW (bioMérieux, Ref. 42043) supplemented with 0.01 g/L of vancomycin (Sigma, Cat. No. 75423).
Sample B: In a homogenizing bag, 25 g of minced steak not contaminated with E. coli O157:H7 are resuspended in 225 ml of BPW (buffered peptone water) supplemented with 0.01 g/L of vancomycin.

The analyses are performed in triplicate for each sample.

The sensitized sponges are immersed in the homogenizing bags before incubation.

The bags are then resealed by means of a closure strip and incubated in a stove at 41.5° C. for 16-24 h.

The bags are then placed in a system allowing compression/decompression of the functionalized sponges throughout incubation (frequency: 1 cycle in 10 s). At the end of the incubation period (20 h at 41.5° C.) the sponges are removed from the enrichment bags and washed in a stomacher bag containing 90 ml of PBS buffer (stomaching for 30 seconds). The aim of this operation is maximum removal of the non-target elements that may be present in the cavities of the sponge.

The presence of the target microorganisms is revealed on the sensitized sponges:

The sponges are brought into contact in a syringe with 500 pl of a conjugated solution of anti-E. coli O157:H7 antibodies labelled with ALP (alkaline phosphatase) for 10 minutes. Washing steps are carried out in PBS-Tween buffer with several piston movements of the syringes.

Contacting with 400 pl of 4-methyl-umbelliferone phosphate substrate in order to reveal the possible presence of ALP conjugate if the test is positive.

Capture of E. coli O157:H7 on the functionalized sponges is verified by the appearance of fluorescence due to the action of the alkaline phosphatase (ALP) present in the conjugate on the substrate.

In this case it is an embodiment for confirming capture of E. coli O157:H7 on the support of the sponge type. The aim is to concentrate the analyte in the course of growth on the sponge in order to reduce the duration of the enrichment step and then proceed to the steps of elution and of detection of the microorganism or microorganisms.

### Example 3

### Capture, Elution and then Detection of Target Microorganisms

A capture substrate, a sponge, consisting of a cube of polyurethane foam, is functionalized with the E. coli O157:H7 phage protein: Sponge “+”.

A non-functionalized polyurethane sponge is also used for evaluating the non-specific capture linked to the structure of the sponge: Sponge “−”.

In a homogenizing bag, 25 g of minced beef are resuspended in 225 ml of buffered peptone water (BPW) preheated to 41.5° C.: negative control.

In another homogenizing bag, 25 g of minced beef artificially contaminated with 100 colony-forming units (CFU) of E. coli O157:H7 are resuspended in 225 ml of BPW (ref. bioMérieux 42043) preheated to 41.5° C.: positive control.

The bags are homogenized for 30 seconds using a stomacher system.

A functionalized sponge “+” and a sponge “−” are placed in two of the bags containing the artificially contaminated minced beef. The sponges are attached to a plastic rod which makes it possible to keep them in the enrichment broth during the incubation and compression/depression time in the system as described in the French patent application filed by the applicant and bearing the filing number 1260566.

The bags are then incubated in an incubator at 41.5° C. and agitated with the system mentioned above enabling the compression/depression of the sponges in the bags (1 cycle every 5 seconds).

At the end of the defined incubation period (in the present case 5 h), the functionalized sponge is removed from the enrichment bag, sprung dry and washed twice with a volume of 5 ml of PBS-Tween 20 at 0.05% in a plastic tube (spin-drying between and after each wash). A volume of 1 ml of PBS buffer is placed in the tube and the latter is placed in a water bath at 100° C. for 10 min to allow the elution of the E. coli O157:H7 cells captured on the sponge substrate.

1.5 ml aliquot fractions of the enrichment broths originating from the control bags not containing sponges (negative and positive bags) are taken after 5 h of enrichment. The samples are placed in a 2 ml Eppendorf tube and heated in a water bath at 100° C. for 10 min.

Using the heated samples (v=500 μl), a VIDAS ECPT (E. coli Phage Technology) test sold by the applicant (ref. 30122)is carried out.

The results obtained in RFV (relative fluorescent value) are noted in Table 1.

TABLE 1
RFV values obtained from the various samples
tested, after 5 h of enrichment

| | RFV value - |
|---|---|
| Sample | VIDAS ECPT |
| Negative control without sponge | −4 |
| Positive control without sponge | 739 |
| Sponge “+” | 1738 |
| Sponge “−” | 5 |

It is seen from the result obtained with the sponges that the sponge “+” has been functionalized and captures the target bacterium (E. coli O157:H7). A positive signal is detected with the VIDAS ECPT kit after washing of the sponges and steps of elution by heating. The levels of RFV signals obtained from the functionalized sponge (and taken up in 1 ml) indicate a concentration of the analyte in comparison with the result obtained from the crude sample (500 μl).

1. A method for capturing and concentrating at least one microorganism or at least one protein secreted by a microorganism that may be present in a sample placed in a container, said method comprising the following steps:
a) in the container, bringing said sample into contact with a culture medium and a sponge capable of capturing the microorganism(s) or the protein(s) secreted by at least one microorganism to be detected, functionalized with a binding partner of at least one microorganism or of at least one secreted protein,
b) placing the container in suitable conditions allowing growth of the microorganism or microorganisms,
c) repeatedly compressing and decompressing the sponge while it remains in contact with said medium.

2. The method according to claim 1, wherein the sponge is immersed in said medium.

3. The method according to claim 1, wherein said medium circulates through the sponge.

4. The method according to claim 1, comprising an additional step consisting of transferring some or all of the mixture consisting of said sample, the culture medium, the sponge and optionally a developer system, from the container, then called the main container, to at least one second container called the secondary container.

5. The method according to claim 1 comprising an additional step of detection of at least one microorganism or of at least one secreted protein bound to the sponge.

6. The method according to claim 5, wherein a developer system able to permit detection is contacted in the main or secondary container prior to the detection step.

7. The method according to claim 6, wherein the detection step is carried out using a means selected from optical detecting means, magnetic detecting means, electrochemical detecting means, electrical detecting means, acoustic detecting means, and thermal detecting means.

8. The method according to claim 6, wherein the developer system is a nonspecific substrate internalized by the microorganism or microorganisms to be detected.

9. The method according to claim 6, wherein the developer system is a cellular stain of the microorganism or microorganisms to be detected.

10. The method according to claim 6, wherein the detection step is carried out directly on the sponge.

11. The method according to claim 10, wherein the sponge is compressed.

12. The method according to claim 1 comprising an additional step of elution of the microorganism or of the secreted protein captured by the sponge.

13. The method according to claim 6, wherein the detection step is carried out in real time.

14. The method according to claim 6, wherein the detection step is carried out at the end of the growth phase of said microorganism or said microorganisms.

15. The method according to claim 1, wherein a binding partner specific or not specific to at least one microorganism or to at least one secreted protein is bound to the sponge.

16. The method according to claim 15, wherein the specific binding partner is selected from a group comprising proteins, antibodies, antigens, phages, phage proteins, aptamers, nucleic acids.

17. The method according to claim 15, wherein the binding partner is bound to the sponge via a dopamine polymer.
